(19) United States
(12) Patent Application Publication
(10) Pub. No.: US 2008/0195178 A1
(43) Pub. Date: Aug. 14, 2008

(54) Fixation methods and systems for cochlear implant component or other implantable devices

(76) Inventor: Janusz A. Kuzma, Parker, CO (US)

Correspondence Address: ADVANCED BIONICS, LLC, 25129 RYE CANYON LOOP, VALENCIA, CA, 91355, US

(21) Appl. No.: 10/987,561

(22) Filed: Nov. 12, 2004

Related U.S. Application Data
(60) Provisional application No. 60/533,399, filed on Dec. 30, 2003.

(51) Int. Cl. A61N 1/00

(52) U.S. Cl. 607/57

(57) ABSTRACT

The present disclosure relates to fixation methods and systems used to secure an implantable medical component in the preferred location of the skull or other bony area of the body. The disclosed fixation methods and systems may be used with a component of a cochlear implant system or other implantable devices, particularly if they are equipped with silicone flaps or flanges, or the like. A mesh reinforcing material overlaps or intertwines into areas of the silicone flanges, which allows for a better distribution of the stress that may occur during the fastening process. Self-tapping screws are used to fasten the implantable component to the skull. The screws are placed in the silicone flanges where the mesh reinforcing material has been embedded. Standard suture-wire may also be used to secure the implantable component to the skull, the implantable component having mounting holes surrounded by the mesh reinforcing material.

The present application claims the benefit of U.S. Provisional Patent Application Ser. No. 60/533,399, filed Dec. 30, 2003, which application is incorporated herein by reference in its entirety.

### BACKGROUND OF THE INVENTION

The present invention relates to the field of implantable medical devices and more particularly to a fixation method used to secure an implantable medical component in the preferred location of the skull or other bony area of the body. The disclosed fixation method may be used with a component of a cochlear implant system or other implantable devices, particularly if they are equipped with silicone flaps or flanges, or the like.

During the surgical procedure of securing a component of an implantable device to a bone, a fixation method is necessary. A variety of these methods exist, for example, one known method is the use of a suture anchor. Such methods are disclosed in U.S. Pat. Nos. 6,106,545; 5,569,303; and 5,807,403; which patents are incorporated herein by reference. The suture anchor methods described in these patents relate to drilling into the bone area, interweaving thread into the drilled channel(s), embedding an anchor into the bone for attaching implantable objects, or adding staples to the bone to secure the suture thread. These methods require intensive drilling into the bone and then interweaving segments of suture thread.

A variety of fixation methods are used in the medical field to secure implantable medical devices. Many of these fixation methods require the step of drilling through the bone area and interweaving a suture thread or wire through the drilled channel(s). Reducing the required steps to secure an implantable component would benefit the surgeon during the implantation procedure.

### SUMMARY OF THE INVENTION

The present disclosure addresses the above and other needs, by providing a fixation method that has the option of using suture-wire or fastening screws to secure the implantable component to the skull or other bony area.

In accordance with one aspect of the disclosure, there is provided a fixation method to secure an implantable component to the skull, while avoiding drilling channels close to the dura mater.

It is a feature of the present disclosure to provide a fixation method that allows for a better distribution of the stress which occurs during the fastening process, by embedding mesh reinforcing material, such as Dacron® fabric, or similar polyester fabric, within the silicone flanges of the implantable component.

It is a further feature of the disclosure to include within the implantable component, mounting holes which aid the surgeon in positioning the implantable component to the skull, and surrounding the mounting holes with Dacron® fabric, or similar polyester fabric, which provides reinforcement and allows for a better distribution of the stress which occurs during the fastening process. The mounting holes can be made during the production process of the implantable component or may be made during the surgical procedure.

It is a further feature of the disclosure to use titanium self-tapping fastening screws or the like to fasten the implantable component to the skull, the implantable component having Dacron fabric embedded around the mounting holes, where the fastening screws would be located.

It is still a further feature of the disclosure to enable the standard suture-wire threading method to mount the implantable component to the skull, the implantable component having Dacron fabric embedded around the mounting holes.

### BRIEF DESCRIPTION OF THE DRAWINGS

The above and other aspects, features and advantages of the present disclosure will be more apparent from the following more particular description thereof, presented in conjunction with the following drawings wherein:

FIG. 1A shows an implantable component mounted on the skull using a suture anchor;

FIG. 1B is a partial top view of the implantable component mounted on the skull shown in FIG. 1A;

FIG. 1C is a partial cross sectional view of the skull taken along line 1C-1C shown in FIG. 1B, where the suture-wire and channel are shown.

FIG. 2A is a perspective view of an implantable component showing the mesh reinforcing material surrounding the mounting holes;

FIG. 2B is a cross sectional view of the implantable component shown in FIG. 2A taken along line 2B-2B;

FIG. 3A shows the implantable component shown in FIG. 2A mounted on the skull using the fixation method described herein;

FIG. 3B is a partial top view of the implantable component mounted on the skull as shown in FIG. 3A;

FIG. 3C is a partial cross sectional view of the implantable component mounted on the skull taken along line 3C-3C shown in FIG. 3B;

FIG. 4A is a partial top view of the implantable component shown in FIG. 2A positioned on the skull and aligned with drilled channels; and

FIG. 4B is a partial cross sectional view of the implantable device shown in FIG. 4A taken along line 4B-4B, where the suture-wire and channel are shown. The suture-wire and knot are simplified and enlarged for purposes of discussion.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

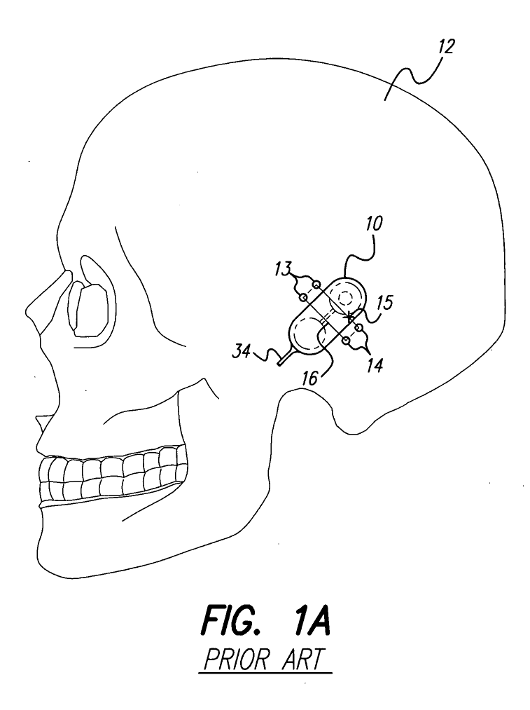

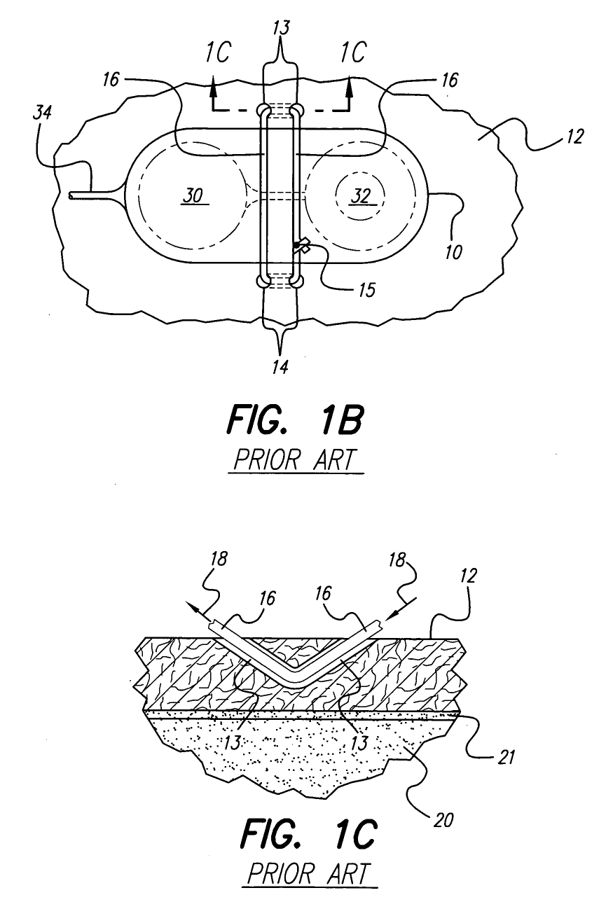

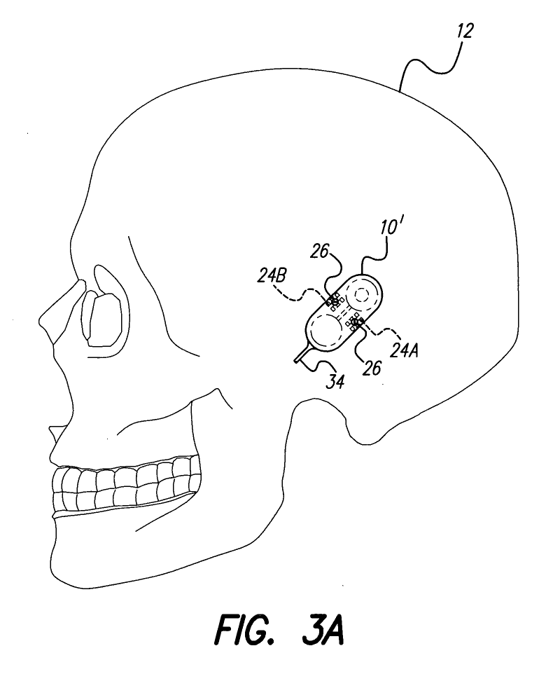

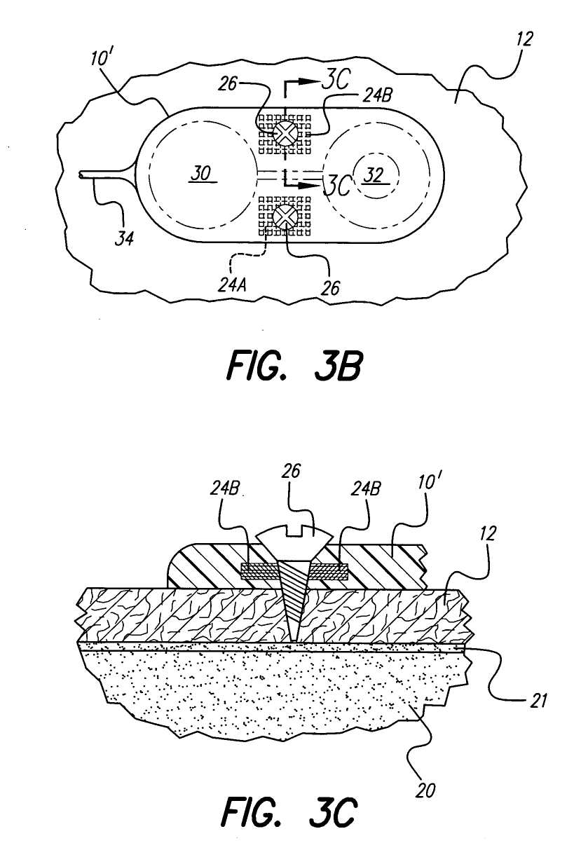

An implantable component 10 of an implantable device mounted on skull 12 is shown in FIG. 1A. The implantable component 10, for instance, can be a part of an implantable cochlear stimulator such as the Clarion® Bionic Ear®, or HiRes 90K cochlear implant systems, which cochlear implant systems are commercially available from Advanced Bionics® Corporation of Sylmar, Calif. The HiRes 90K ICS is described, e.g., in U.S. Pat. No. 6,219,580, incorporated herein by reference. Any cochlear implant system may benefit from the present disclosure, as well as other implantable devices. The Clarion Bionic Ear, and HiRes 90K cochlear implant systems are referenced herein as examples of how the best mode of the disclosure may be implemented. An individual component of the cochlear prostheses, such as the implantable pulse generator (IPG), may be the implantable component 10 that would be mounted to the skull 12, as shown in FIG. 1A, or the implantable component 10′ shown in FIG. 3A. The mounting process would allow a stable position for the implantable component 10 or 10′ where the associated electrode lead 34 would also remain stable.

Several fixation methods exist in the art that suit the needs of securing the implantable device in the preferred location. One such method that is known in the art is the use of a suture anchoring method, as shown in FIG. 1A. This type of anchoring method includes the steps of initially preparing/drilling two parallel channels in the bone of the skull; inserting a fixation suture-wire or thread into the first channel; threading the suture-wire from the first channel over the implantable component; threading the suture-wire into the second channel; and securing both ends of the suture-wire with a knot.

FIG. 1B is a partial top view of the implantable component 10 positioned in a section of the skull 12. The implantable component 10 is placed between the two formed parallel channels, the first channel 13 and the second channel 14. The first channel 13 and the second channel 14 are drilled into the skull 12 as shown in FIG. 1C, where FIG. 1C is a sectional view taken along line 1C-1C shown in FIG. 1B. Channels 13 and 14 are drilled into the thickness of the skull 12, while avoiding penetrating the dura mater 21 and brain area 20. Suture-wire 16 is threaded into the first channel 13 as shown in FIG. 1C and as indicated by the arrow 18. It is then threaded into the second channel 14 over the implantable component 10 as shown in FIG. 1B. Both ends of the suture-wire 16 are made into a knot 15, as is known in the art of securing suture-wire.

The suture anchor method described above is a multi-step process that carries the risk of accidentally drilling the channels 13 and 14 too deep, potentially damaging the brain area 20 and/or dura mater 21, a risk that the patient and the surgeon would rather avoid. Another potential risk involves having the implantable component 10 become loose, possibly requiring repositioning. Yet another possibility is the suture, and especially the knot 15 of the suture-wire 16, eroding through the skin or causing some other irritation. The need for an improved, more secure method for fixating the implantable component 10 is the primary focus of the present disclosure.

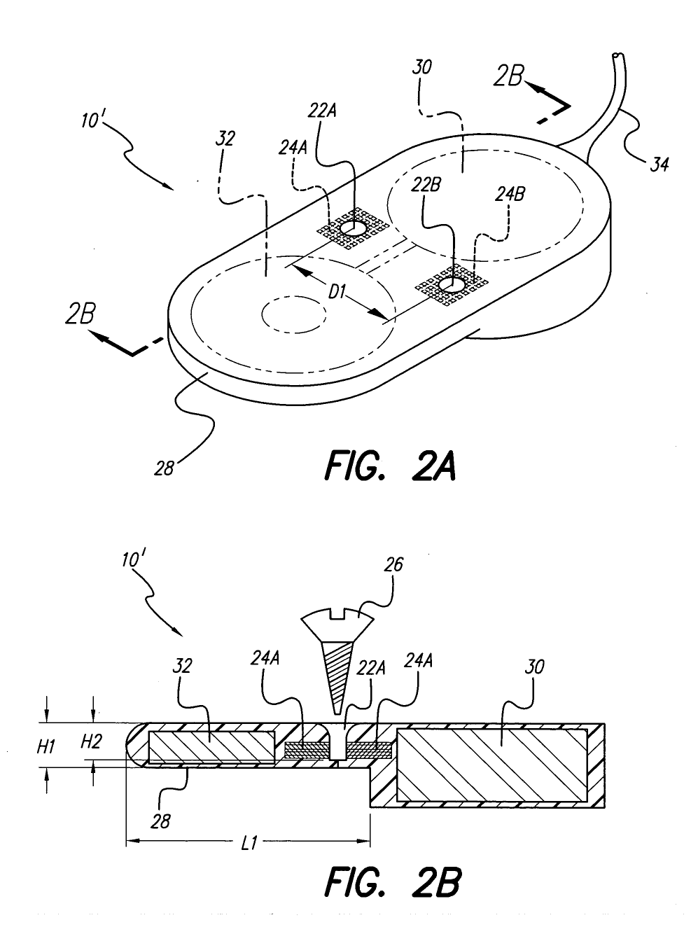

Turning next to FIG. 2A, an implantable component 10′, such as an implantable pulse generator (IPG), is shown. During the molding process in which element 30 and element 32 of the IPG are embedded within a silicone material or other biocompatible material, two or more mounting holes, 22A and 22B, can be created or such holes can be created during the surgical procedure. The mounting holes 22A and 22B can be spaced apart a distance D1. Pieces of a mesh reinforcing material, such as Dacron® fabric 24A and 24B or similar polyester fabric are positioned within the silicone material surrounding the mounting holes 22A and 22B. The pieces of fabric 24A and 24B can also be placed in other areas of the implantable device, in a silicone or other biocompatible material which may be formed via molding or other suitable method and the mounting holes can be created during surgery. Therefore, where, herein, reference is made to silicone material, it is understood that other suitable biocompatible material(s) may be used, and where reference is made to molding the material, it is understood that any other suitable process may be used, and these variations are encompassed by the present disclosure. The pieces of Dacron fabric 24A and 24B reinforce and provide strength to the silicone material in the flap section 28 of the IPG or implantable component 10′. The pieces of Dacron fabric 24A and 24B allow for a better distribution of stress during the fixation process.

Referring now to FIG. 2B, a cross sectional view of the implantable component 10′ taken along line 2B-2B, is shown. The exemplary implantable component 10′ has a flap section 28, with a thickness H1 and length L1. The mounting holes 22A and 22B are molded in the area of flap section 28, away from the embedded elements 30 and 32 of the IPG. The mounting holes 22A and 22B can be molded through the thickness H1, through a thickness H2 which is slightly less than H1, or created during surgery. Molding the holes 22A and 22B with a height H2, allows some material to remain in the mounting hole, which may result in a more secure connection between the silicone flap 28 and the fastening screw 26. A titanium self-tapping screw 26, having a round head or flat head may be used to fasten the implantable component 10′ onto the skull 12. Other types of fasteners may be used that are known in the art for fixating implantable devices into bone tissue.

FIG. 3A shows the implantable component 10′ or IPG fastened to the skull 12 using titanium self-tapping screws 26. The length of the self-tapping screw is selected so that it penetrates into the thickness H1 of the silicone flap 28 and it also penetrates into some thickness of the skull, with enough length to provide a secure fit while avoiding piercing the dura mater 21. As shown in FIG. 3A, two titanium self-tapping screws 26 are used, but only one or more than two screws may be used, for instance, if required for secure positioning of the implantable component 10′.

A top partial view of the implantable component 10′ is shown in FIG. 3B which also shows the possible locations of the fastening screws 26. The fastening screws 26 are secured in the silicone area where pieces of the Dacron fabric 22A and 22B have been embedded and away from the embedded elements 30 and 32. FIG. 3C, which is a partial cross sectional view of FIG. 3B taken along line 3C-3C, shows a titanium self-tapping screw 26 secured into the skull tissue, while avoiding penetrating the dura mater 21. The pieces of Dacron fabric 22A and 22B provide strength to the silicone material in the flap section 28. During the process of securing the implantable component 10′ to the skull, the Dacron fabric also allows for a better distribution of the stress over the fastening area of the silicone material.

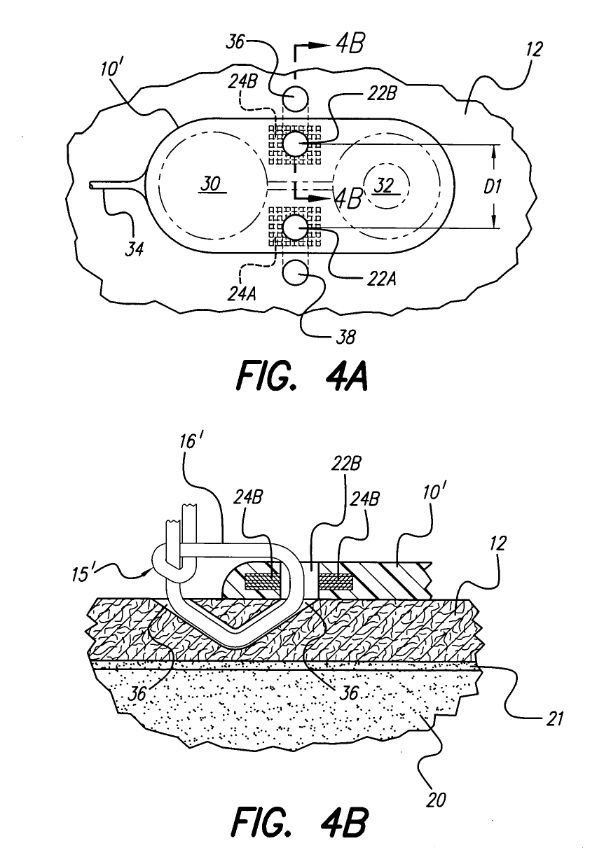

Another fixation method is shown in FIG. 4A. The implantable component 10′ is placed on the skull aligning the mounting holes 22A and 22B with channels 36 and 38. Channels 36 and 38 have been previously drilled in an aligned configuration during the surgical procedure and spaced apart a distance D1 similar to the distance between the mounting holes 22A and 22B. As shown in FIG. 4B, suture-wire 16′ is threaded into channel 36 and through mounting hole 22B. Both ends of the suture-wire 16′ are made into a knot 15′, as it is known in the art of securing suture-wire. Another piece of suture-wire, not shown in FIG. 4B, is threaded through channel 38 and mounting hole 22A where both ends of the suture-wire are also made into a knot. This fixation method uses the standard channel drilling process in the skull 12, but secures an implantable component 10′ which contains Dacron fabric or other type of polyester fabric within the silicone mold. The added strength which the fabric provides to the mounting holes 22A and 22B allows for a secure coupling between the skull 12 and the implantable component 10′.

While the invention herein disclosed has been described by means of specific embodiments and applications thereof, numerous modifications and variations could be made thereto by those skilled in the art without departing from the scope of the invention set forth in the claims.

1. An implantable medical device, comprising:
biocompatible material enclosing components used in the treatment of a patient;
reinforcing material embedded in at least a portion of the biocompatible material; and
at least one fastener penetrating the reinforcing material embedded in the biocompatible material;
wherein the at least one fastener comprises at least one self-tapping screw configured to secure the implantable medical device to bone.

2. The implantable medical device of claim 1 wherein the implantable medical device comprises a component of a cochlear implant system.

3. The implantable medical device of claim 2 wherein the component of the cochlear implant system comprises an implantable pulse generator.

4. The implantable medical device of claim 1 wherein the biocompatible material is silicone.

5. The implantable medical device of claim 1 wherein the reinforcing material is a polyester fabric.

6. The implantable medical device of claim 5 wherein the polyester fabric is mesh.

7. The implantable medical device of claim 1 wherein the at least one self-tapping screw comprises titanium.

8. The implantable medical device of claim 1 wherein the at least one fastener is configured to secure the implantable medical device to the skull.

9. The implantable medical device of claim 1 wherein the biocompatible material and the reinforcing material overlap or intertwine and have at least one hole passing therethrough through which the at least one fastener may be positioned.

10. A method of securing an implantable medical device to bone, comprising:
enclosing components of the implantable medical device within a biocompatible material;
embedding a reinforcing material in at least a portion of the biocompatible material;
penetrating the reinforcing material embedded in the biocompatible material with at least one fastener; and
securing the implantable medical device to bone with the at least one fastener, wherein the at least one fastener comprises at least one screw and wherein the securing step comprises screwing at least one of said at least one screws into bone.

11. The method of claim 10 wherein the implantable medical device comprises a component of a cochlear implant system.

12. The method of claim 11 wherein the component of the cochlear implant system comprises an implantable pulse generator.

13. The method of claim 10 wherein forming the implantable medical device comprises molding.

14. The method of claim 10 wherein the biocompatible material is silicone.

15. The method of claim 10 wherein the reinforcing material is a polyester fabric.

16. The method of claim 15 wherein the polyester fabric is mesh.

17. The method of claim 10 wherein the at least one screw comprises at least one titanium self-tapping screw.

18. The method of claim 10 wherein enclosing the implantable medical device and embedding the reinforcing material in at least a portion of the biocompatible material further comprises forming at least one hole in which the at least one fastener may be positioned.

19. A method of securing an implantable medical device to bone, comprising:
enclosing components of the implantable medical device within a biocompatible material;
embedding a reinforcing material in at least a portion of the biocompatible material;
forming at least one hole through the reinforcing material;
forming at least a first channel in the bone;
securing the implantable medical device to the bone with suture-wire positioned through the at least one hole in the implantable medical device and through the at least first channel in the bone.

20. The method of claim 19 wherein the implantable medical device comprises a component of a cochlear implant system.

21. The method of claim 20 wherein the component of the cochlear implant system comprises an implantable pulse generator.

22. The method of claim 19 further including forming a second channel in the bone that is substantially parallel to the at least first channel.

23. The method of claim 19 wherein forming the implantable medical device comprises molding.

24. The method of claim 19 wherein the reinforcing material is a polyester fabric.
